(19) United States
(12) Patent Application Publication
(10) Pub. No.: US 2026/0011454 A1
(43) Pub. Date: Jan. 8, 2026

(54) SYSTEMS AND METHODS FOR PROVIDING CUSTOMIZED CONVERSATIONAL SUPPORT BETWEEN A PATIENT AND A CAREGIVER

(71) Applicants: Edmund LOFTUS, Gainesville, FL (US); Edison LOFTUS, Gainesville, FL (US)

(72) Inventors: Edmund LOFTUS, Gainesville, FL (US); Edison LOFTUS, Gainesville, FL (US)

(21) Appl. No.: 19/159,008

(22) PCT Filed: Feb. 7, 2024
(86) PCT No.: PCT/US2024/014728

§ 371 Date: Aug. 22, 2025

Related U.S. Application Data
(60) Provisional application No. 63/483,662, filed on Feb. 7, 2023.

(51) Int. Cl. G16H 80/00; G10L 15/18; G10L 15/22; G10L 15/30

(52) U.S. Cl. CPC G16H 80/00; G10L 15/1815; G10L 15/22; G10L 15/30

(57) ABSTRACT

The subject invention pertains to a conversational management system including an enhanced chatbot (ECB) configured to enhance a repetitive communication between a caregiver and a patient. The system can provide a multiplicity of topics extracted from a dialogue between the patient and the caregiver, prompts extracted from the topics, responses derived from the topics, an ECB script that connects the prompts to the responses, an ECB audio database that contains the responses in a voice recognizable to the patient as a trusted voice. The invention can further provide a patient-optimized voice response software application configured and adapted to deliver selected content from the ECB audio database according to the ECB script, thus supporting a prompt-response dialogue with the patient to enhance the repetitive communication between the caregiver and the patient. Also, a method for enhancing a unidirectional communication between a caregiver and a patient.

### CROSS-REFERENCE TO RELATED APPLICATION

The present application claims the benefit of U.S. Provisional Application Ser. No. 63/483,662, filed Feb. 7, 2023, which is hereby incorporated by reference herein in its entirety, including any figures, tables, nucleic acid sequences, amino acid sequences, or drawings.

### BACKGROUND OF THE INVENTION

As noted by the Center for Disease Control and Prevention in their Alzheimer's Disease and Healthy Aging website (https://www.cdc.gov/aging/aginginfo/alzheimers.htm), Alzheimer's disease is the most common type of dementia. In 2020, as many as 5.8 million Americans were living with Alzheimer's disease. In addition to memory problems, someone with symptoms of Alzheimer's disease can experience numerous symptoms and challenges, including repeating questions. Currently, many people living with Alzheimer's disease are cared for at home by family members. Caregiving can have positive aspects for the caregiver as well as the person being cared for. It can bring personal fulfillment to the caregiver, such as satisfaction from helping a family member or friend, and lead to the development of new skills and improved family relationships.

Although most people willingly provide care to their loved ones and friends, caring for a person with Alzheimer's disease at home can be a difficult task and can become overwhelming at times. Each day brings new challenges as the caregiver copes with changing levels of ability and new patterns of behavior. As the disease gets worse, people living with Alzheimer's disease often need more intensive care.

One common area of challenge for caregivers is the need to support the patient through periods of repetitive conversation, repeated questions, and recurring themes in dialogue. On the other hand, at certain stages a dementia patient could not wish to or not be able to engage in a conversation about their feelings, but they still can be suffering from intense anxiety caused by delusion or confusion. Under these circumstances, a patient can suffer from a lack of ability or desire to engage in bidirectional communication that could help reduce or mitigate their delusion or confusion.

### BRIEF SUMMARY OF THE INVENTION

Embodiments of the subject invention provide systems and methods for providing individually customized conversational support on a conversation (e.g., phone call, video call, web chat, or other communication) between a patient (e.g., a person, diagnosed or undiagnosed, presenting symptoms consistent with Alzheimer's, dementia, senility, or the like) and a caregiver (e.g., a clinician, physician, nurse, gerontologist, care-facility staff member, therapist, family member, spouse, or loved one).

In cases where a dementia patient does not wish to or is not able to engage in a conversation about their feelings, but they still can be suffering from intense anxiety caused by delusion or confusion, embodiments of the subject invention can provide benefit in the form of a reassuring message (e.g., in the voice of a trusted loved one) that can specifically mitigate or redirect the patient's concern away from the delusion or confusion with reduced demands of engaging in bidirectional conversation for the patient, the caregiver, or both. An example of this messaging in certain embodiments can include when a patient is becoming anxious about a previously-identified primary topic (alternatively a nonspecific topic) but is unable or unwilling to engage in a dialogue, a caregiver can deliver a unidirectional primary (alternatively, nonspecific) response in the voice of a trusted loved one. This unidirectional primary (alternatively, nonspecific) response can provide some or all of the benefits provided by a bidirectional chatbot, to either encourage a positive emotional reaction or mitigate a negative emotional reaction.

In certain embodiments a generative AI application can be used to compose a primary (alternatively, nonspecific) response by incorporating elements of the primary (alternatively, nonspecific) topic of concern to the patient into the AI prompt. Then, the AI-generated primary (alternatively, nonspecific) response text can be edited for content and either recorded or synthesized in the voice of a trusted loved one so that a caregiver can play the primary (alternatively, nonspecific) response for a patient when they are feeling concerned about a primary (alternatively, nonspecific) topic.

In certain embodiments a caregiver and a patient begin a call via telephone or other electronic communication means. The caregiver adds an enhanced chatbot (ECB) according to an embodiment of the subject invention to the call at their discretion. Alternatively, the ECB can be added automatically or upon recognition of certain predefined criteria including but not limited to identification of certain phone numbers, network addresses, communication sources, time of day, days of the week, content of the conversation, speech patterns, volume level, or tone. The ECB provides personalized conversational support throughout the call. The ECB comprises a list of prompts (e.g., known phrases, recorded clips, or parameterized analytic speech profiles) expected from the patient. Each prompt can be matched to one or more responses. Responses can be recorded, simulated, or synthesized in the voice of the caregiver or another trusted voice. Responses can be customized to the needs of the patient and can be selected to favor positive reinforcement, reassurance, and calming thoughts for the patient (e.g., I love you, Things will be alright, We're getting better, etc.) By parameterized analytic speech profiles is meant any portion of speech determined to match or be appropriate for a given prompt, situation, or usage case based on an analysis (e.g., including one or more of an objective assessment, or a subjective assessment) of one or more parameters defined to categorize speech. Examples include but are not limited to analysis of a numeric, binary, scalar, or rubric-based parameter related to speech intensity, pace, tone, volume, a combination of any of the foregoing, and/or a change in any of the foregoing. By way of non-limiting examples, parameterized analytic speech profiles can include one or more segments or components of speech selected for their increasing measures of both pace and volume, for a decrease in tone at a constant volume, or for an increase in intensity matched with a decrease in pace.

Embodiments provide responses optimized to address repetitive delusions, anxieties, fears, stories, concerns, questions, and need for reassurance that are often associated with Alzheimer's, senility, dementia, and other aging, behavioral, psychological, or cognitive diseases, disorders, syndromes, symptoms, or behaviors.

In certain embodiments, the ECB can be configured to provide natural dialogue transitions (e.g., “OK”, “Go on.”, “I′m listening.”, etc.) whenever a prompt from the patient is noticed without a response from the caregiver (e.g., within a specified time limit, or following a specific prompt, pattern of prompts, or lack of prompts.)

Embodiments comprising the ECB can provide a list of generic responses to provide when the patient's words do not match any prompt or meet another predetermined criteria (e.g., no response, “grunt”, “sigh”, “mmm”, “I′m sorry”, “What was that?”, “Could you say that again?”, etc.)

The ECB can have a list of redirecting responses for known sticking points (e.g., a commonly recurring prompt from the patient related to sadness and missing a deceased pet can be mapped to response(s) referencing a current pet in a positive context, “Well at least we still have Spot. He sure loves to play in the yard . . . ”)

If multiple responses are matched to a recognized prompt, a first response can be chosen randomly, sequentially, or by another method. The random response function is greatly beneficial since a singular response to a highly-repeated patient prompt can result in abnormal dialogue patterns, breaks, or loops in the conversation. Responses can be rated or categorized along one or more parameters including, for example, positivity, reactivity, seasonality, or a record of prior response from one or more patients. Embodiments of the ECB can be configured and adapted to build a database of response data while serving patients and to apply the data collected to improve services provided to patients.

The caregiver can go on mute, or listen passively, then jump back in when needed. This passive interaction of the caregiver can be greatly beneficial since caregiver fatigue can lead to stress and anxiety for the caregiver and reduced quality of care for the patient.

The caregiver can do other work (or rest/recover mentally) while the ECB provides reassurance and connection for the patient. Further, the ECB can be configured to defer whenever the caregiver talks over, interrupts, or jumps in. The ECB can be advantageously configured to always (alternatively, preferentially, predominantly, or within specified parameters) defer to the caregiver, so that the caregiver can step in at any time to avoid a negative experience or frustration for the patient. The caregiver can provide feedback and edit the prompts and responses to improve ECB customization for a specific patient. The ECB can also collect performance or response data to learn what works for an individual patient, a specified group of patients, or all patients within a given category, demographic, geography, or other defining feature. For example, the ECB can record conversational data related to one or more prompts in male patients as compared to female patients; patients in different cities, states, or countries; or patients having various vocational (e.g., teacher, engineer, physician, mechanic, or construction worker) or avocational (e.g., artist, fisherman, cyclist, or baseball fan) backgrounds or experiences. Embodiments can provide patient profiles and/or caregiver profiles to advantageously organize and analyze one or more demographic factors useful in providing improved conversational support.

The caregiver can initiate and conclude conversations normally, and use the ECB support for expected, recurring, or perceived highly repetitive portions of the conversation. Alternatively, the caregiver can use the ECB support to advantageously provide a structured, reliable, and beneficial scripted initiation or conclusion (or both) to repetitive conversations with one or more patients. Embodiments of the ECB can advantageously include scripted implementations of conversation in accordance with, following, or informed by guidelines for patient care and management from a healthcare provider or organization (e.g., the National Institutes of Health, the World Health Organization, or a personal physician, social worker, nurse, or psychiatrist of the patient.) Embodiments of the ECB can be provided as a service associated with or supported by a medical practice, hospital, or other organization to better support patients.

One exemplary and non-limiting embodiment of the subject invention comprises the following features and steps:
1. Caregiver and patient begin call
2. Caregiver adds ECB to call
3. ECB provides personalized conversational support
   3.1. ECB comprises a list of prompts (e.g., known phrases, recorded clips, or parameterized analytic speech profiles) expected from the patient
      3.1.1. Each prompt can be matched to one or more responses
      3.1.2. Responses can be recorded, simulated, or synthesized in the voice of the caregiver
      3.1.3. Responses can be customized to the needs of the patient
      3.1.4. Responses can be selected to favor positive reinforcement, reassurance, and calming thoughts for the patient (e.g., “I love you”, “Things will be alright”, “We're getting better”, etc.)
      3.1.5. Responses can be optimized to address repetitive delusions, anxieties, fears, stories, concerns, questions, and need for reassurance that are often associated with Alzheimer's and dementia.
      3.1.6. ECB can be configured to provide natural dialogue transitions (e.g., “OK”, “Go on.”, “I'm listening.”, etc.) whenever a prompt from the patient is noticed without a response from the caregiver (within a specified time limit)
   3.2. ECB can have a list of generic responses to provide when the patient's words do not match any prompt (e.g., no response, “I′m sorry”, “What was that?”, “Could you say that again?”, etc.)
   3.3. ECB can have a list of redirecting responses for known sticking points (e.g., a commonly recurring prompt from the patient related to sadness and missing a deceased pet can be mapped to response(s) referencing a current pet in a positive context, “Well at least we still have Spot. He sure loves to play in the yard . . . ”)
   3.4. If multiple responses are matched to a recognized prompt, a first response can be chosen randomly, sequentially, or by another method. The random response function is greatly beneficial, since a singular response to a highly-repeated patient prompt can result in abnormal dialogue patterns or loops.
4. Caregiver can go on mute, or listen passively, then jump back in when needed (this passive interaction of the caregiver is greatly beneficial since caregiver fatigue can lead to stress and anxiety for the caregiver and reduced quality of care for the patient.)
5. Caregiver can do other work (or rest/recover mentally) while ECB provides reassurance and connection for the patient
6. ECB can be configured to defer whenever the caregiver talks over, interrupts, or jumps in
7. ECB can be advantageously configured to always (alternatively, preferentially, or within specified parameters) defer to the caregiver, so that the caregiver can step in at any time to avoid a negative experience or frustration for the patient
8. Caregiver can provide feedback and edit the prompts and responses to improve ECB customization for a specific patient
9. Caregiver can initiate and conclude conversations normally, and use the ECB support for the highly repetitive portions.

### BRIEF DESCRIPTION OF THE DRAWINGS

FIG. 1 illustrates a process flow for ECB creation and ECB use according to an embodiment of the subject invention.

FIG. 2 illustrates a detailed process flow for passive monitoring according to an embodiment of the subject invention.

FIG. 3 depicts a graphical taxonomy or relationship diagram of components of dialogue between patient and caregiver, including primary topics, secondary topics, pauses in dialogue, primary prompts, secondary prompts, pause detection, primary responses, secondary responses, default responses, encouraging positive emotional reactions, mitigating negative emotional reactions, sustaining dialogue, active listening in a secondary response, encouraging dialogue, and active listening in a default response, according to an embodiment of the subject invention.

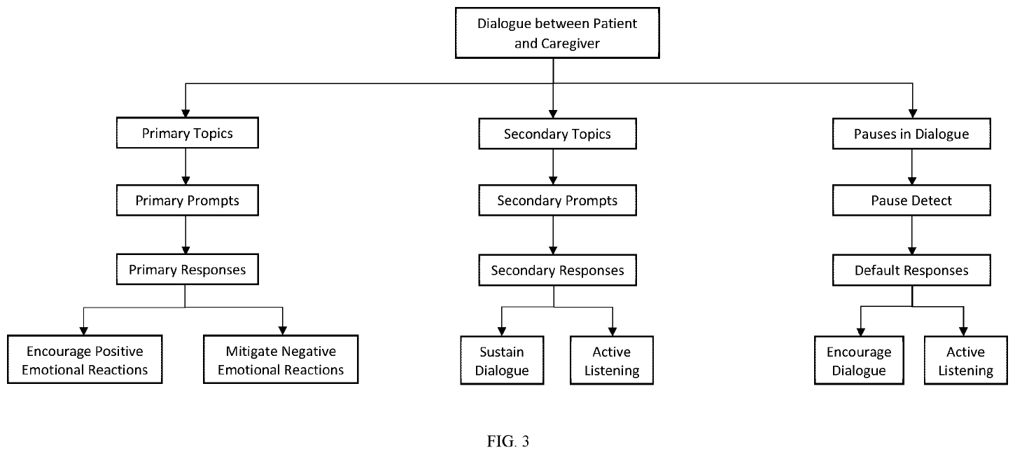

FIG. 4 is a schematic showing how the telephonic voice connections between the caregiver and patient interface with the Google Dialogflow application in an initial prototype embodiment of the subject invention.

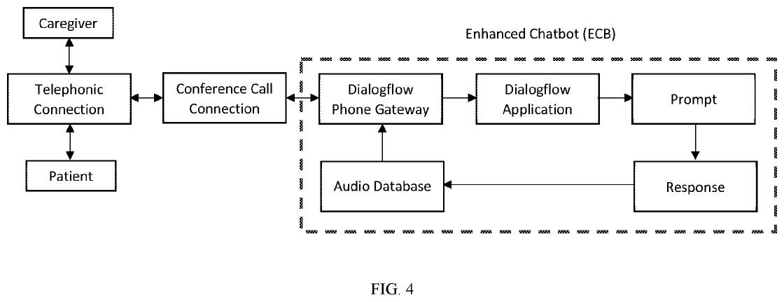

FIG. 5 illustrates a process flow for AI message creation and AI message use according to an embodiment of the subject invention.

### DETAILED DESCRIPTION OF THE INVENTION

Embodiments of the subject invention provide a conversational management system configured and adapted to enhance a repetitive telephonic communication by providing customized conversational support between a caregiver and a patient.

The patient can be any person displaying symptoms of dementia, memory loss, or similar conditions that interfere with their ability to sustain coherent dialogue. Examples include individuals suffering from Alzheimer's Disease, dementia, Lewy Body Dementia, or other ailments and can be diagnosed or undiagnosed.

The caregiver can be any person who provides supportive accommodations to a patient. Examples include a spouse, a child, a sibling, or a health care professional engaged to support a patient.

The repetitive telephonic communication can include any communication where at least one party repeats or returns to a topic, a viewpoint, an observation, or a series of words or phrases more than once, and can occur over any technology that transmits and receives audible voices. Examples include landline telephone, mobile phone, voice over IP (VOIP) chat service, online forum, mobile application, virtual reality experiences, or other verbal communication media.

Suitable conversational management systems can include any technology configurable to provide automated responses optimized to meet unique patient conversational needs. One exemplary and non-limiting embodiment of a system according to the subject invention has been implemented by the inventors using Dialogflow (https://cloud.google.com/dialogflow; Google, Mountain View, CA). Dialogflow can operate in multiple modalities, including texting, phone calls, and web applications. Dialogflow is commonly used to provide enterprise customer support services including virtual chatbots, which are formulaic applications. However, Dialogflow supports custom audio creation and response randomization, which can each, respectively, alone or in combination, be advantageously employed in certain embodiments of the subject invention. Additional commercial platforms are available and contemplated within the scope of certain embodiments of the subject invention, including IBM Watson Assistant, Amazon Lex, and Microsoft Bot Framework. Future developments in technology, features, availability, commercial or proprietary access, modes of operation, and application programming interfaces are anticipated and expected in this field. Future improvements in these and other infrastructure or support services are contemplated within the scope of and expected to be applied within certain embodiments of the subject invention.

In certain embodiments the system comprises a multiplicity of topics that are extracted from a dialogue between the patient and the caregiver, wherein the multiplicity of topics comprises a set of primary topics that are repetitive and specific to the patient, and a set of secondary topics that can be repetitive but are non-specific (e.g., less intensely impactful, reactionary, triggering, or upsetting) to the patient. By primary topics (e.g., topics that are specific to the patient), is meant topics that are known or expected to produce a strong, consistent, and/or repetitive response in the patient. By secondary topics (e.g., including topics that are non-specific to the patient), is meant topics that are known or expected to produce a mild, inconsistent, or non-repetitive response in the patient. By repetitive is meant topics that the patient persistently repeats or returns to even in cases where they have already been addressed by the caregiver within the dialogue period.

Primary topics and secondary topics relevant to a given patient can often be discovered, developed, or categorized through observation and/or interaction with the patient. Such observation and/or interaction can be in-person or remote, manual or automated, and can be based on methods including but not limited to a discovery script or protocol, a flow chart, passive monitoring, analysis of past conversations, interviews, surveys, or questionnaires with the patient, caregiver, or third parties, qualitative or quantitative analysis, and other methods known in the art.

The dialogue period can be defined in certain embodiments as a single continuous conversation, a phone call, a period of time such as one day, one hour, 5 minutes, 2 minutes, 1 minute, or a thread of conversation having contextual continuity with or without temporal continuity. Unusually high frequencies of repetition can be defined as either (i) High Repetition: Patient repeats topics more than once within five minutes and repetitions are not consecutive; or (ii) Very High Repetition: Patient repeats topics three or more times consecutively.

In the context of the subject invention, topics are subjects of verbal conversation that are recognizable and meaningful to the patient in a manner that stimulates predictable responses by the patient.

Primary topics specific to the patient include, for example, subjects that patients have a strong desire to discuss and are distinguished by being frequently repeated and the triggering of predictable and often intense emotional reactions, both positive and negative. Positive reactions include but are not limited to joy, relief, appreciation, recognition, affirmation, affection, sympathy, and confidence. Negative reactions include fear, anger, grief, despair, distrust, betrayal, and apathy.

Importance to the patient is defined by the extent to which a particular subject is repeated and elicits a strong emotional response. Subjects that are frequently discussed and are associated with strong emotional reactions are of greater importance to patients in the context of certain embodiments of the subject invention.

Secondary topics non-specific to the patient include:
i.Basic information gathering or clarifying questions (e.g., those beginning with: Who, What, When, Where, Why, How, Are, Do, Is)
ii. Basic declarative statements of a personal or general nature (e.g., “I don't know”, “That's not true”, “I didn't say that.”, etc.)
iii. Transitional phrases that are part of natural spoken language (e.g., “OK”, “Really?”, “Yeah”, etc.)

Secondary topics are distinguished by being unrelated to, distinct, or disconnected from the majority or all of the primary topics.

Topics non-specific to the patient are also distinguishable by their general, random or transitional nature.

Topics can be extracted by manual or automated methods. Such methods can include distillation of notes taken by a caregiver or third party after participating in conversations with a patient. Such methods can further include computer-compiled themes associated with pre-compiled or specifically generated sets of topics, that are triggered by keyword filters or generated by artificial intelligence (AI) algorithms from a recorded or real-time conversations with a patient.

A dialogue between a patient and a caregiver can be a weekly phone call, an unscheduled conversation, or any other model of verbal interaction. A dialogue can occur in any physical location and over any medium capable of supporting verbal communication via landline telephone, mobile phone, voice over IP (VOIP) chat service, online forum, mobile application, virtual reality experiences, or other verbal communication media. A dialogue can include in-person conversation while stationary or riding in a car, bus, plane, or other vehicle. A dialogue can include live or recorded video, avatars, metaverse characters, or an audio-only dialogue without a substantive visual representation.

In certain embodiments the system comprises a multiplicity of prompts extracted from the multiplicity of topics, the multiplicity of prompts originating from the patient and comprising a set of primary prompts from the set of primary topics, and a set of secondary prompts from the set of secondary topics.

Prompts comprise, consist of, or consist essentially of singular words, groups of words, or complete sentences spoken by a patient that are indicative of primary or secondary topics. A known characteristic of dementia and similar conditions is that a patient will often want to discuss a limited scope of topics with a high degree of repetition and/or emotional intensity. This type of conversational drive can be the result of the delusions, hallucinations, and memory loss associated with dementia and similar conditions, whereby reasoning and information retention have been significantly impaired. This smaller, repetitive, and unique scope of topics can be represented by prompts, which form the basis for dialogue sustained by embodiments of the subject invention.

In certain embodiments the system comprises a multiplicity of responses derived from the multiplicity of topics, the multiplicity of responses originating from ECB and comprising a set of primary responses mapped to the set of primary prompts, a set of secondary responses mapped to the set of secondary prompts, and default responses that occur when the system does not recognize any primary or secondary prompts in the patient's locution.

Primary responses are highly customized to the needs of the patient and are based on optimal communication techniques that have been observed and/or developed between the patient and caregiver. Primary responses are designed to encourage positive emotional reactions and mitigate negative emotional reactions.

Secondary responses help sustain system dialogue with the patient and provide an active listening experience for the patient.

Default responses comprise, consist of, or consist essentially of periods of silence that occur after phrases spoken by the patient, which the system does not recognize as primary or secondary prompts. The presence of these silent default responses contributes to the experience of active listening. Default responses also comprise, consist of, or consist essentially of transitional phrases or words such as “OK”, “Go on”, “Yeah”, or other non-comital phrases, which when paired with one or more periods of silence, can help encourage further dialogue.

An ECB script can connect the set of primary prompts to the set of primary responses and can connect the set of secondary prompts to the set of secondary responses. An ECB audio database can contain the set of primary responses and the set of secondary responses in a voice recognizable to the patient as a trusted voice. A patient-optimized voice response software application can be configured and adapted to deliver selected content from the ECB audio database according to the ECB script, thus supporting a prompt-response dialogue with the patient to enhance the repetitive telephonic communication between the caregiver and the patient.

An embodiment of the ECB script can comprise, consist of, or consist essentially of a series of tabular entries, for example, including Title, Prompts, Responses, and Audio Database sections. The title can be a distilled description of a group of prompts that are specific to the patient. Responses can be comprised of one or more entries that are able to encourage a positive emotional reaction and/or mitigate a negative emotional reaction from the patient. The audio database can contain the names of the audio files corresponding to the trusted-voice responses. Table 1 shows an excerpt from an exemplary functional script according to an embodiment of the subject invention.

TABLE 1
Example Script Segment

| | |
|---|---|
| Title | I don't know . . . |
| Prompts | I don't know what to do. |
| | I don't know who else to talk to. |
| | I don't know who to go to. |
| | I need someone to help me figure this out. |
| Responses | Me and Ryan will help you mom. |
| | You can always talk to me about it. |
| | Ryan knows, and he's helping |
| | Don't worry mom, me and Ryan are looking for all those people who took the |
| | money. |
| Audio | meandryanwillhelp.wav |
| Database | talktomeaboutit.wav |
| | ryanknow.wav |

A complete script can comprise, consist of, or consist essentially of several segments that can be organized in the format of Table 1. The script content is then loaded into a voice response software application that facilitates an automated prompt-response dialogue optimized for the needs of a particular patient.

In another embodiment the system comprises an AI application that responds directly to patient-initiated topics in the trusted voice of the caregiver. The AI application learns to provide patient-optimized responses (e.g., by being trained with effective patient-caregiver dialogues.) In this manner, successful responses for encouraging positive emotional reactions, mitigating negative emotional reactions, and facilitating natural dialogue can be learned and automatically delivered by the AI application.

Embodiments provide a set of primary responses associated with encouraging a positive emotional reaction and/or mitigating a negative emotional reaction including two or more techniques selected from the group consisting of understanding, redirection, and positive reinforcement. In certain embodiments these and other techniques are applied in ways that are most meaningful to the patient. For example, if a patient is expressing a perspective that is not based in reality, a caregiver can encourage a positive emotional reaction and/or mitigate a negative emotional reaction by responding that they understand the patient. Sometimes a patient can repeatedly express feeling of sadness or anger; in these situations, a caregiver can choose to redirect the conversation by inserting a completely unrelated, but positive, response into the conversation. The effects of short-term memory loss can cause or allow the patient to forget about the negative topic and focus on the positive redirection. Sometimes a patient can need assurances and affirmation about something positive (e.g., needing to hear that they were a good parent or spouse during their lives.) Hearing these affirmations can bring comfort to a patient. In this context, the caregiver can express positive reinforcement, thereby substantiating the patient's positive feeling and encouraging a positive emotional reaction. For a patient with memory issues, these techniques can need to be repeated, for example, 5-10 times per topic within the bounds of a single conversation that can last 30-60 minutes, alternatively 10-120 minutes, or 20-90 minutes. Alternatively, a conversation in the context of certain embodiments of the subject invention can last more than 1, 5, 10, 20, 30, 60, 90, or 120 minutes, including ranges, combinations, and increments of any of the foregoing. Alternatively, a conversation in the context of certain embodiments of the subject invention can last less than 1, 5, 10, 20, 30, 60, 90, 120, 150, or 180 minutes, including ranges, combinations, and increments of any of the foregoing. These patients often forget that they've had these conversations, and will want to repeat the same conversations many (e.g., 10 or more, alternatively 2 or more, alternatively 2-9, including ranges, combinations, and increments of any of the foregoing) times per day. Embodiments of the subject invention offer an automated system and/or method for delivering conversational techniques that encourage positive emotional reactions and/or mitigate negative emotional reactions in an efficient and effective manner that relieves burden and strain from the caregiver.

Embodiments provide a set of secondary responses comprising active listening techniques such as clarification, repetition, and confirmation. For example, a patient can respond well if the caregiver asks the patient to explain something further. This keeps the patient in control of the conversation and gives them an opportunity to say more about what might be important to them at that moment. Similarly, if the caregiver inserts phrases such as “Go on!” or “Really?” into a conversation, the patient can thereby receive feedback that helps encourage additional dialogue. Patients will often ask if the caregiver is there or if they can be heard, and in these situations it is effective for the caregiver to confirm that yes, they're present and listening. A patient can also ask if they're being understood, and a caregiver can reply to confirm that the caregiver is there and did hear what the patient said. For a patient with memory issues, these techniques are frequently needed to maintain dialogue about a topic or transition to a new topic. Embodiments of the subject invention offer an automated system and method for delivering conversational techniques that provide an experience of active listening by a caregiver in an efficient and effective manner that relieves burden and strain from the caregiver.

Embodiments provide a set of primary responses and a set of secondary responses each, respectively, comprising either interactive or automated input, or both, from the caregiver. Interactive input can comprise, consist of, or consist essentially of live verbal communication from the caregiver to the patient, as in a typical telephonic conversation. Automated input can comprise, consist of, or consist essentially of responses from the audio database delivered by the system according to the response-prompt correlation established in the script. In one embodiment of the invention, a caregiver begins a conversation with a patient by participating in normal dialogue with the patient on either an outgoing or incoming telephonic communication, which is an example of interactive input from the caregiver. After establishing the dialogue, the caregiver can engage the system to provide automated input of responses from the audio database according to the response-prompt correlations established in the script. The system can be connected to a caregiver-patient conversation by means of a three-way calling function, interactive communication application, hosted cloud service, or other connection mechanism. During this time, the caregiver can passively monitor the conversation while on mute, while the system continues to provide automated input. The caregiver can unmute at any time to guide to conversation, check on the patient, or conclude the conversation. In this manner, the system functions with both interactive and automated inputs within a single conversation.

In another embodiment of the invention, a patient can connect with the system directly using a telephonic communication system, and the automated inputs from the system initiate, sustain, and conclude the conversation automatically, with no interaction by the caregiver.

In certain embodiments, a caregiver can engage in conversation with a patient that consists primarily, essentially, or exclusively of interactive input.

Embodiments provide a set of primary responses comprising interactive input from the caregiver. In certain embodiments, the set of primary responses consist essentially of automated input from the caregiver (e.g., responses recorded, synthesized, created, or modeled from recordings or characteristics of the caregiver, to address primary topics related to the patient, then categorized, organized, and stored by the ECB for automatic delivery at a prescribed time or based on a prescribed pattern or occurrence of events such as one or more primary prompts), and the set of secondary responses consist essentially of automated input from the caregiver (e.g., responses recorded, synthesized, created, or modeled from recordings or characteristics of the caregiver, to address secondary topics related to the patient, then categorized, organized, and stored by the ECB for automatic delivery at a prescribed time or based on a prescribed pattern or occurrence of events such as one or more secondary prompts.)

In certain embodiments, the trusted voice comprises, consists of, or consists essentially of one or more mannerisms derived from the patient, a family member of the patient, or a friend of the patient; the one or more mannerisms comprising, consisting of, or consisting essentially of at least one of tone, cadence, intensity, regional dialect, accent, or cultural reference. These mannerisms are vocal patterns that make a voice or pattern of speech recognizable to a patient, and that recognition is often associated with a relationship that the patient trusts. A known characteristic of dementia and similar conditions is that a patient will often lose both trust in and recognition of people over time, even family members and those that they have been close to for years. Configuring the system to provide an automated communication experience with a recognized and trusted voice can optimize the effectiveness of conversational techniques that encourage positive emotional reactions and/or mitigate negative emotional reactions from the patient.

In certain embodiments, the trusted voice is the voice of the caregiver or an approximation thereof. The patient-optimized voice response software application can comprise a telephone user interface and a response randomizing function. The response randomizing function can be configured and adapted to provide a different response selected from the set of primary responses to each of two or more successive prompts selected from the set of primary prompts. In this manner, the response randomizing function provides non-repetitive replies to the patient's inputs. This facilitates more natural dialogue and inhibits the conversation going into a loop, whereby something the patient says causes the system to repeat the same response continuously, for a period of time, or until a negative or unnatural development in or conclusion of the conversation.

Embodiments provide a method for enhancing a repetitive telephonic communication between a caregiver and a patient having a need for dialogue. The method can include initiating, by the caregiver, before or during the repetitive telephonic communication, a conversational management system configurable to provide automated dialogue optimized to meet unique patient conversational needs; muting, by the caregiver, to interrupt audio input from the conversational management system to the patient, or from caregiver to the patient; engaging, by the conversational management system, a prompt-response dialogue with the patient, whereby patient verbal prompts are associated with optimized audio responses stored in the conversation management system; monitoring, by the caregiver, the prompt-response dialogue between the conversational management system and the patient such that the caregiver can engage the automated dialogue with interactive input if needed; discerning, by the caregiver, an insufficient response from the conversational management system to the patient (e.g. patient expresses confusion about the automated dialogue or asks a direct question that does not have a corresponding automated response); unmuting, by the caregiver, following the insufficient response, to permit audio input from the caregiver to the patient; and providing, by the caregiver, following the insufficient response, one or more redirecting responses to the patient that address confusion or questions and allows continuation of the automated dialogue.

Embodiments can further include muting again, by the caregiver, after giving the one or more redirecting responses to the patient, to interrupt audio input from the caregiver and allow resumption of the prompt-response dialogue between the conversational management system and the patient; monitoring again, by the caregiver after muting again, the prompt-response dialogue between the conversational management system and the patient such that the caregiver can re-engage the automated dialogue with interactive input if needed; determining, by the caregiver, a desired time to end the prompt-response dialogue between the conversational management system and the patient based on time constraints of one or both parties or cues from the patient that the conversation is reaching a natural conclusion; unmuting again, by the caregiver, to permit audio input from the caregiver to the patient; and providing, by the caregiver, one or more terminating responses to the patient that will conclude the communication.

Embodiments can further include muting again, by the caregiver, after giving the one or more redirecting responses to the patient, to interrupt audio input from the caregiver and allow resumption of the prompt-response dialogue between the conversational management system and the patient; monitoring again, by the caregiver after muting again, the prompt-response dialogue between the conversational management system and the patient such that the caregiver can re-engage the automated dialogue with interactive input if needed; determining, by the conversational management system, based at least in part on one or more prompts from the patient, a desired time to end the prompt-response dialogue between the conversational management system and the patient; providing, by the conversational management system, one or more terminating responses to the patient; and terminating, by the conversational management system, the repetitive telephonic communication with the patient.

Embodiments can further include a first repetition of a first primary prompt spoken by the patient and recognized by the conversational management system from a list of primary prompts; a first primary response played back by the conversational management system in answer to the first repetition of the first primary prompt; a second repetition of the first primary prompt spoken by the patient and recognized by the conversational management system from the list of primary prompts; a second primary response played back by the conversational management system in answer to the second repetition of the first primary prompt; the second primary response being different from the first primary response.

Embodiments can further include the prompt-response dialogue comprising: a first repetition of a first secondary prompt spoken by the patient and recognized by the conversational management system from a list of secondary prompts; a first secondary response played back by the conversational management system in answer to the first repetition of the first secondary prompt; a second repetition of the first secondary prompt spoken by the patient and recognized by the conversational management system from the list of secondary prompts; a second secondary response played back by the conversational management system in answer to the second repetition of the first secondary prompt; the second secondary response being different from the first secondary response.

Embodiments provide a system for providing patient-centric, caregiver-supportive, customized conversational support to a conversation between a patient having needs including a need for repetitive dialogue and a caregiver having a desire to support the conversational needs of the patient. The system can comprise a database comprising a multiplicity of primary prompts expected from the patient, a multiplicity of primary responses linked to each respective primary prompt, and list of default responses not linked to any primary prompt, wherein each respective primary prompt is linked to more than one primary response, and wherein each respective primary response is linked to at least one respective primary prompt and is optimized to support the needs of the patient in a context related to the respectively linked primary prompt, and a processor; and a machine-readable medium in operable communication with the processor and having instructions stored thereon that, when executed by the processor, perform the following steps: initiating or joining a conversation between the patient and the caregiver; iteratively monitoring the conversation for dialogue from the patient and for each occurrence of a primary prompt from the patient, then following each respective primary prompt: checking for a response from the caregiver, when a response from the caregiver is detected following a respective primary prompt, deferring to the caregiver and then returning to iteratively monitoring, when no response from the caregiver is detected following a respective primary prompt, delivering a primary response to the patient within a natural flow of the conversation, such that the conversation is maintained with reduced effort from the caregiver, and then returning to iteratively monitoring; and when dialogue from the patient is detected in the absence of a primary prompt, and in the absence of a response from the caregiver, delivering a default response to the patient within a natural flow of the conversation, such that the conversation is maintained with reduced effort from the caregiver, and then returning to iteratively monitoring; thereby providing the patient-centric, caregiver-supportive, customized conversational support to the conversation between the patient and the caregiver.

Embodiments can further provide a system wherein the multiplicity of primary prompts expected from the patient are derived from or expressed as known phrases, recorded clips, or parameterized analytic speech profiles expected from the patient; the multiplicity of primary responses linked to each respective primary prompt are derived from or embodied in a form comprising at least one of a voice, a mannerism, or an image recognized by the patient as a trusted source; and the delivering of each primary response comprises selecting the primary response randomly, sequentially, or by another method that reduces a perception of repetition in the patient.

Embodiments can further provide a system wherein each respective primary response is optimized by inclusion of one or more characteristics selected from the list consisting of active listening, understanding, redirection, and positive reinforcement.

Embodiments can further provide a system wherein each respective primary response is optimized by inclusion of one or more words, phrases, expressions, or topics correlated to a positive response from the patient through manual or automated analysis of one or more current or prior conversations including the patient or including another individual matched to the patient by at least one demographic or metric.

Embodiments can further provide a system wherein the instructions, when executed by the processor, perform the following steps: detecting a termination signal from the caregiver; determining if the termination signal indicates a passive termination, or an active termination; if the termination signal indicates a passive termination, pausing for a predetermined period of time to allow the caregiver to terminate the conversation; if the termination signal indicates an active termination, executing a predetermined or adaptive prompt-response dialogue designed to naturally end the conversation with the patient.

Embodiments of the subject invention address the technical problem of providing conversational support to a patient having a need for repetitive dialogue (e.g., diagnosed or undiagnosed symptoms consistent with Alzheimer's Disease, dementia, age-related memory loss, or other ailments) being difficult, mentally and emotionally demanding, time consuming, and challenging for family, healthcare providers, or caregivers. This problem is addressed by providing conversational support in the form of an enhanced chatbot (ECB), in which an ECB script is utilized to advantageously provide optimized responses to certain prompts identified from the patient. Provided responses can include automated responses that can be recorded, simulated, or synthesized in, replicating, approximating, or suggesting the voice of the caregiver or another trusted voice. Responses can be customized to the needs of the patient and can be selected to favor, encourage, or provide positive reinforcement, reassurance, and calming thoughts for an individual patient or a demographic group of patients. Responses can also be selected to minimize, discourage, or avoid topics associated with negative reactions, negative experiences, frustration, anger, or obsession for an individual patient or a demographic group of patients.

By way of illustration and not limitation, the following non-limiting exemplary embodiments are offered:

Embodiment 1. A conversational management system configured and adapted to enhance a repetitive communication between a caregiver and a patient, the system comprising:
a multiplicity of topics extracted from a dialogue between the patient and the caregiver, the multiplicity of topics comprising:
   a set of primary topics that are repetitive and important to the patient, and
   a set of secondary topics that are repetitive and non-specific to the patient;
a multiplicity of prompts extracted from the multiplicity of topics, the multiplicity of prompts originating from the patient and comprising:
   a set of primary prompts related to the set of primary topics, and
   a set of secondary prompts related to the set of secondary topics;
a multiplicity of responses derived from the multiplicity of topics, the multiplicity of responses originating from an enhanced chatbot (ECB) and comprising:
   a set of primary responses mapped to the set of primary prompts, and
   a set of secondary responses mapped to the set of secondary prompts;
an ECB script that connects the set of primary prompts to the set of primary responses and connects the set of secondary prompts to the set of secondary responses;
an ECB audio database that contains the set of primary responses and the set of secondary responses in a voice recognizable to the patient as a trusted voice; and
a patient-optimized voice response software application configured and adapted to deliver selected content from the ECB audio database according to the ECB script, thus supporting a prompt-response dialogue with the patient to enhance the repetitive communication between the caregiver and the patient.

Embodiment 2. The system according to Embodiment 1, the set of primary responses including two or more characteristics selected from the group consisting of active listening, understanding, redirection, and positive reinforcement.

Embodiment 3 The system according to Embodiment 2, the set of secondary responses comprising clarification, repetition, and confirmation.

Embodiment 4. The system according to Embodiment 1, the set of primary responses and the set of secondary responses each, respectively, comprising either interactive or automated input, or both, from the caregiver.

Embodiment 5. The system according to Embodiment 4, the set of primary responses comprising interactive input from the caregiver.

Embodiment 6. The system according to Embodiment 1, the set of primary responses consisting essentially of automated input from the caregiver, and the set of secondary responses consisting essentially of automated input from the caregiver.

Embodiment 7. The system according to Embodiment 1, wherein the trusted voice comprises one or more mannerisms derived from the patient, a family member of the patient, or a friend of the patient; the one or more mannerisms comprising at least one of cadence, intensity, regional dialect, accent, and cultural reference.

Embodiment 8. The system according to Embodiment 7, wherein the trusted voice is the voice of the caregiver.

Embodiment 9. The system according to Embodiment 1, wherein the patient-optimized voice response software application comprises a telephone user interface and a response randomizing function.

Embodiment 10. The system according to Embodiment 9, wherein the response randomizing function is configured and adapted to provide a different response selected from the set of primary responses to each of two successive prompts selected from the set of primary prompts.

Embodiment 11. A method for creating a conversational management system configured and adapted to support a prompt-response dialogue with a patient to enhance a repetitive telephonic communication between a caregiver and a patient, the method comprising:
identifying a multiplicity of topics during a dialogue between the patient and the caregiver;
identifying a set of primary topics that are repetitive and important to the patient;
identifying a set of secondary topics that are repetitive and non-specific to the patient;
extracting a set of primary prompts from the set of primary topics;
extracting a set of secondary prompts from the set of secondary topics;
establishing a set of primary responses mapped to the set of primary prompts;
establishing a set of secondary responses mapped to the set of secondary prompts;
creating an enhanced chatbot (ECB) script that connects the set of primary prompts to the set of primary responses and connects the set of secondary prompts to the set of secondary responses;
creating an ECB audio database that contains the set of primary responses and the set of secondary responses in a voice recognizable to the patient as a trusted voice; and
loading the ECB script and ECB audio database into a patient-optimized voice response software application, thereby providing the conversational management system configured and adapted to support the prompt-response dialogue with the patient to enhance the repetitive telephonic communication between the caregiver and the patient.

Embodiment 12. The method according to Embodiment 11, the set of primary responses including two or more characteristics selected from the group consisting of active listening, understanding, redirection, and positive reinforcement.

Embodiment 13. The method according to Embodiment 12, the set of secondary responses comprising clarification, repetition, and confirmation.

Embodiment 14. The method according to Embodiment 11, the establishing of the set of primary responses and the establishing of the set of secondary responses each, respectively, comprising either interactive or automated input, or both, from the caregiver.

Embodiment 15. The method according to Embodiment 14, the establishing of the set of primary responses comprising interactive input from the caregiver.

Embodiment 16. The method according to Embodiment 11, the establishing of the set of primary responses consisting essentially of automated input from the caregiver, and the establishing of the set of secondary responses consisting essentially of automated input from the caregiver.

Embodiment 17. The method according to Embodiment 11, wherein the trusted voice comprises one or more mannerisms derived from the patient, a family member of the patient, or a friend of the patient; the one or more mannerisms comprising at least one of cadence, intensity, regional dialect, accent, and cultural reference.

Embodiment 18. The method according to Embodiment 17, wherein the trusted voice is the voice of the caregiver.

Embodiment 19. The method according to Embodiment 11, wherein the patient- optimized voice response software application comprises a telephone user interface and a response randomizing function.

Embodiment 20. The method according to Embodiment 19, wherein the response randomizing function is configured and adapted to provide a different response selected from the set of primary responses to each repetition of a prompt selected from the set of primary prompts.

Embodiment 21. A method for enhancing a repetitive telephonic communication between a caregiver and a patient having a need for dialogue, the method comprising:
initiating, by the caregiver, before or during the repetitive telephonic communication, a conversational management system;
muting, by the caregiver, an audio input to the repetitive telephonic communication;
engaging, by the conversational management system, a prompt-response dialogue with the patient;
monitoring, by the caregiver, the prompt-response dialogue between the conversational management system and the patient;
determining, by the caregiver, an insufficient response from the conversational management system to the patient;
unmuting, by the caregiver, following the insufficient response, the audio input to the repetitive telephonic communication; and
providing, by the caregiver, following the insufficient response, one or more redirecting responses to the patient.

Embodiment 22. The method according to Embodiment 21, comprising:
muting again, by the caregiver, after giving the one or more redirecting responses to the patient, the audio input to the repetitive telephonic communication;
monitoring again, by the caregiver after muting again, the prompt-response dialogue between the conversational management system and the patient;
determining, by the caregiver, a desired time to end the prompt-response dialogue between the conversational management system and the patient;
unmuting again, by the caregiver, the audio input to the repetitive telephonic communication; and
providing, by the caregiver, one or more terminating responses to the patient.

Embodiment 23. The method according to Embodiment 21, comprising:
muting again, by the caregiver after giving the one or more redirecting responses to the patient, the audio input to the repetitive telephonic communication;
monitoring again, by the caregiver after muting again, the prompt-response dialogue between the conversational management system and the patient;
determining, by the conversational management system, based at least in part on one or more prompts from the patient, a desired time to end the prompt-response dialogue between the conversational management system and the patient;
providing, by the conversational management system, one or more terminating responses to the patient; and
terminating, by the conversational management system, the repetitive telephonic communication with the patient.

Embodiment 24. The method according to Embodiment 21, the prompt-response dialogue comprising:
a first repetition of a first primary prompt spoken by the patient and recognized by the conversational management system from a list of primary prompts;
a first primary response played back by the conversational management system in answer to the first repetition of the first primary prompt;
a second repetition of the first primary prompt spoken by the patient and recognized by the conversational management system from the list of primary prompts; and
a second primary response played back by the conversational management system in answer to the second repetition of the first primary prompt;
the second primary response being different from the first primary response.

Embodiment 25. The method according to Embodiment 24, the prompt-response dialogue comprising:
a first repetition of a first secondary prompt spoken by the patient and recognized by the conversational management system from a list of secondary prompts;
a first secondary response played back by the conversational management system in answer to the first repetition of the first secondary prompt;
a second repetition of the first secondary prompt spoken by the patient and recognized by the conversational management system from the list of secondary prompts; and
a second secondary response played back by the conversational management system in answer to the second repetition of the first secondary prompt;
the second secondary response being different from the first secondary response.

Embodiment 26. A system for providing patient-centric, caregiver-supportive, customized conversational support to a conversation between a patient having needs including a need for repetitive dialogue and a caregiver having a desire to support the needs of the patient, the system comprising:
a database comprising a multiplicity of primary prompts expected from the patient, a multiplicity of primary responses linked to each respective primary prompt, and list of generic responses not linked to any primary prompt, wherein each respective primary prompt is linked to more than one primary response, and wherein each respective primary response is linked to at least one respective primary prompt and is optimized to support the needs of the patient in a context related to the respectively linked primary prompt;
a processor; and
a machine-readable medium in operable communication with the processor and having instructions stored thereon that, when executed by the processor, perform the following steps:
initiating or joining a conversation between the patient and the caregiver;
iteratively monitoring the conversation for dialogue from the patient and for each occurrence of a primary prompt from the patient, then following each respective primary prompt:
checking for a response from the caregiver,
when a response from the caregiver is detected following a respective primary prompt, deferring to the caregiver and then returning to iteratively monitoring,
when no response from the caregiver is detected following a respective primary prompt, delivering a primary response to the patient within a natural flow of the conversation, such that the conversation is maintained with reduced effort from the caregiver, and then returning to iteratively monitoring; and
when dialogue from the patient is detected in the absence of a primary prompt, and in the absence of a response from the caregiver, delivering a generic response to the patient within a natural flow of the conversation, such that the conversation is maintained with reduced effort from the caregiver, and then returning to iteratively monitoring;
thereby providing the patient-centric, caregiver-supportive, customized conversational support to the conversation between the patient and the caregiver.

Embodiment 27. The system according to Embodiment 26, wherein:
the multiplicity of primary prompts expected from the patient are derived from or expressed as known phrases, recorded clips, or parameterized analytic speech profiles expected from the patient;
the multiplicity of primary responses linked to each respective primary prompt are derived from or embodied in a form comprising at least one of a voice, a mannerism, and an image recognized by the patient as a trusted source; and
the delivering of each primary response comprises selecting the primary response randomly, sequentially, or by another method that reduces a perception of repetition in the patient.

Embodiment 28. The system according to Embodiment 26, wherein each respective primary response is optimized by inclusion of one or more characteristics selected from the group consisting of active listening, understanding, redirection, and positive reinforcement.

Embodiment 29. The system according to Embodiment 26, wherein each respective primary response is optimized by inclusion of one or more words, phrases, expressions, or topics correlated to a positive response from the patient through manual or automated analysis of one or more current or prior conversations including the patient or including another individual matched to the patient by at least one demographic or metric.

Embodiment 30. The system according to Embodiment 26, wherein the instructions, when executed by the processor, perform the following steps:
detecting a termination signal from the caregiver;
determining if the termination signal indicates a passive termination, or an active termination;
if the termination signal indicates a passive termination, pausing for a predetermined period of time to allow the caregiver to terminate the conversation;
if the termination signal indicates an active termination, executing a predetermined or adaptive prompt-response dialogue designed to naturally end the conversation with the patient.

Embodiment 31. A unidirectional communication management system configured and adapted to enhance a positive communication from a caregiver to a patient, the system comprising:
a multiplicity of topics extracted from a dialogue between the patient and the caregiver, the multiplicity of topics comprising:
   a set of primary topics that are recurring and important to the patient, and
   a set of secondary topics that are recurring and non-specific to the patient;
a multiplicity of responses derived from the multiplicity of topics, the multiplicity of responses originating from an enhanced chatbot (ECB) and comprising:
   a set of primary responses mapped to the set of primary topics, and
   a set of secondary responses mapped to the set of secondary topics;
an ECB script that connects the set of primary topics to the set of primary responses and connects the set of secondary topics to the set of secondary responses;
an ECB audio database that contains the set of primary responses and the set of secondary responses in a voice recognizable to the patient as a trusted voice; and
a patient-optimized voice response software application configured and adapted to deliver selected content from the ECB audio database according to the ECB script, thus delivering a unidirectional positive reinforcement communication to the patient.

Embodiment 32. The system according to Embodiment 31, the set of primary responses including two or more characteristics selected from the group consisting of active listening, understanding, redirection, and positive reinforcement.

Embodiment 33. The system according to Embodiment 32, the set of secondary responses comprising clarification, repetition, and confirmation.

Embodiment 34. The system according to Embodiment 31, the set of primary responses and the set of secondary responses each, respectively, comprising either interactive or automated input, or both, from the caregiver.

Embodiment 35. The system according to Embodiment 34, the set of primary responses comprising interactive input from the caregiver.

Embodiment 36. The system according to Embodiment 31, the set of primary responses consisting essentially of automated input from the caregiver, and the set of secondary responses consisting essentially of automated input from the caregiver.

Embodiment 37. The system according to Embodiment 31, wherein the trusted voice comprises one or more mannerisms derived from the patient, a family member of the patient, or a friend of the patient; the one or more mannerisms comprising at least one of cadence, intensity, regional dialect, accent, and cultural reference.

Embodiment 38. The system according to Embodiment 37, wherein the trusted voice is the voice of the caregiver.

Embodiment 39. The system according to Embodiment 31, wherein the patient-optimized voice response software application comprises a telephone user interface and a response randomizing function.

Embodiment 40. The system according to Embodiment 39, wherein the response randomizing function is configured and adapted to provide a different response selected from the set of primary responses to each of two successive occurrences of a topic selected from the set of primary topics.

Embodiment 41. A method for creating a unidirectional communication management system configured and adapted to enhance a positive communication from a caregiver to a patient, the method comprising:
identifying a multiplicity of topics during a dialogue between the patient and the caregiver;
identifying a set of primary topics that are repetitive and important to the patient;
identifying a set of secondary topics that are repetitive and non-specific to the patient;
establishing a set of primary responses mapped to the set of primary topics;
establishing a set of secondary responses mapped to the set of secondary topics;
creating an enhanced chatbot (ECB) script that connects the set of primary topics to the set of primary responses and connects the set of secondary topics to the set of secondary responses;
creating an ECB audio database that contains the set of primary responses and the set of secondary responses in a voice recognizable to the patient as a trusted voice; and
loading the ECB script and ECB audio database into a patient-optimized voice response software application, thereby providing the conversational management system configured and adapted to deliver the unidirectional communication to the patient to enhance the positive communication from the caregiver to the patient.

Embodiment 42. The method according to Embodiment 41, the set of primary responses including two or more characteristics selected from the group consisting of active listening, understanding, redirection, and positive reinforcement.

Embodiment 43. The method according to Embodiment 42, the set of secondary responses comprising clarification, repetition, and confirmation.

Embodiment 44. The method according to Embodiment 41, the establishing of the set of primary responses and the establishing of the set of secondary responses each, respectively, comprising either interactive or automated input, or both, from the caregiver.

Embodiment 45 The method according to Embodiment 44, the establishing of the set of primary responses comprising interactive input from the caregiver.

Embodiment 46. The method according to Embodiment 41, the establishing of the set of primary responses consisting essentially of automated input from the caregiver, and the establishing of the set of secondary responses consisting essentially of automated input from the caregiver.

Embodiment 47. The method according to Embodiment 41, wherein the trusted voice comprises one or more mannerisms derived from the patient, a family member of the patient, or a friend of the patient; the one or more mannerisms comprising at least one of cadence, intensity, regional dialect, accent, and cultural reference.

Embodiment 48. The method according to Embodiment 47, wherein the trusted voice is the voice of the caregiver.

Embodiment 49. The method according to Embodiment 41, wherein the patient-optimized voice response software application comprises a telephone user interface and a response randomizing function.

Embodiment 50. The method according to Embodiment 49, wherein the response randomizing function is configured and adapted to provide a different response selected from the set of primary responses to each repetition of a prompt selected from the set of primary prompts.

Embodiment 51. A method for enhancing a unidirectional communication between a caregiver and a patient having a need for reassurance, the method comprising:
initiating, by the caregiver, before or during the unidirectional communication, a conversational management system;
muting, by the caregiver, an audio input to the unidirectional communication;
engaging, by the conversational management system, a unidirectional communication to the patient;
monitoring, by the caregiver, the unidirectional communication from the conversational management system to the patient;
determining, by the caregiver, an insufficient response from the conversational management system to the patient;
unmuting, by the caregiver, following the insufficient response, the audio input to the unidirectional communication; and
providing, by the caregiver, following the insufficient response, one or more redirecting responses to the patient.

Embodiment 52. The method according to Embodiment 51, comprising:
muting again, by the caregiver, after giving the one or more redirecting responses to the patient, the audio input to the unidirectional communication;
monitoring again, by the caregiver after muting again, the prompt-response dialogue between the conversational management system and the patient;
determining, by the caregiver, a desired time to end the unidirectional communication from the conversational management system to the patient;
unmuting again, by the caregiver, the audio input to the unidirectional communication; and
providing, by the caregiver, one or more terminating responses to the patient.

Embodiment 53. The method according to Embodiment 51, comprising:
muting again, by the caregiver after giving the one or more redirecting responses to the patient, the audio input to the unidirectional communication;
monitoring again, by the caregiver after muting again, the unidirectional communication from the conversational management system to the patient;
determining, by the conversational management system, a desired time to end the unidirectional communication from the conversational management system to the patient;
providing, by the conversational management system, one or more terminating responses to the patient; and
terminating, by the conversational management system, the unidirectional communication to the patient.

Embodiment 54. A method for enhancing direct playing of audio of a caregiver to a patient having a need for reassurance, the method comprising:
initiating, by the caregiver, before or during the direct playing of audio, a conversational management system;
muting, by the caregiver, an audio input to the direct playing of audio;
engaging, by the conversational management system, a unidirectional communication to the patient;
monitoring, by the caregiver, the unidirectional communication from the conversational management system to the patient;
determining, by the caregiver, an insufficient response from the conversational management system to the patient;
unmuting, by the caregiver, following the insufficient response, the audio input to the direct playing of audio; and
providing, by the caregiver, following the insufficient response, one or more redirecting responses to the patient.

Embodiment 55. The method according to Embodiment 54, comprising:
muting again, by the caregiver, after giving the one or more redirecting responses to the patient, the audio input to the direct playing of audio;
monitoring again, by the caregiver after muting again, the prompt-response dialogue between the conversational management system and the patient;
determining, by the caregiver, a desired time to end the direct playing of audio from the conversational management system to the patient;
unmuting again, by the caregiver, the audio input to the direct playing of audio; and
providing, by the caregiver, one or more terminating responses to the patient.

Embodiment 56. The method according to Embodiment 54, comprising:
muting again, by the caregiver after giving the one or more redirecting responses to the patient, the audio input to the direct playing of audio;
monitoring again, by the caregiver after muting again, the direct playing of audio from the conversational management system to the patient;
determining, by the conversational management system, a desired time to end the direct playing of audio from the conversational management system to the patient;
providing, by the conversational management system, one or more terminating responses to the patient; and
terminating, by the conversational management system, the direct playing of audio to the patient.

Embodiment 57. The system according to Embodiment 31, wherein the patient-optimized voice response software application is based on a telephony system.

Embodiment 58. The method according to Embodiment 41, wherein the conversational management system is based on a telephony system.

Embodiment 59. The method according to Embodiment 51, wherein the conversational management system is based on a telephony system.

Embodiment 60. The method according to Embodiment 54, wherein the conversational management system is based on a telephony system.

Embodiment 61. The system according to Embodiment 51, further comprising a generative AI application configured to generate a primary response by incorporating elements of the set of primary topics into an AI prompt, wherein the AI-generated primary response is configured for content and either recorded or synthesized in a voice of the caregiver and/or a trusted loved one of the patient, and wherein the caregiver plays the AI-generated primary response to the patient.

Embodiment 62. The method according to Embodiment 54 further comprising configuring a generative AI application to generate a primary response by incorporating elements of the set of primary topics into an AI prompt, wherein the AI-generated primary response is configured for content and either recorded or synthesized in a voice of the caregiver and/or a trusted loved one of the patient, and wherein the caregiver plays the AI-generated primary response to the patient.

Embodiment 63. The system according to Embodiment 1, wherein the patient-optimized voice response software application is based on a telephony system.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consist essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

It should be understood that the examples and embodiments described herein are for illustrative purposes only and that various modifications or changes in light thereof will be suggested to persons skilled in the art and are to be included within the spirit and purview of this application and the scope of the appended claims.

### EXAMPLES

### Example 1: Functional Script

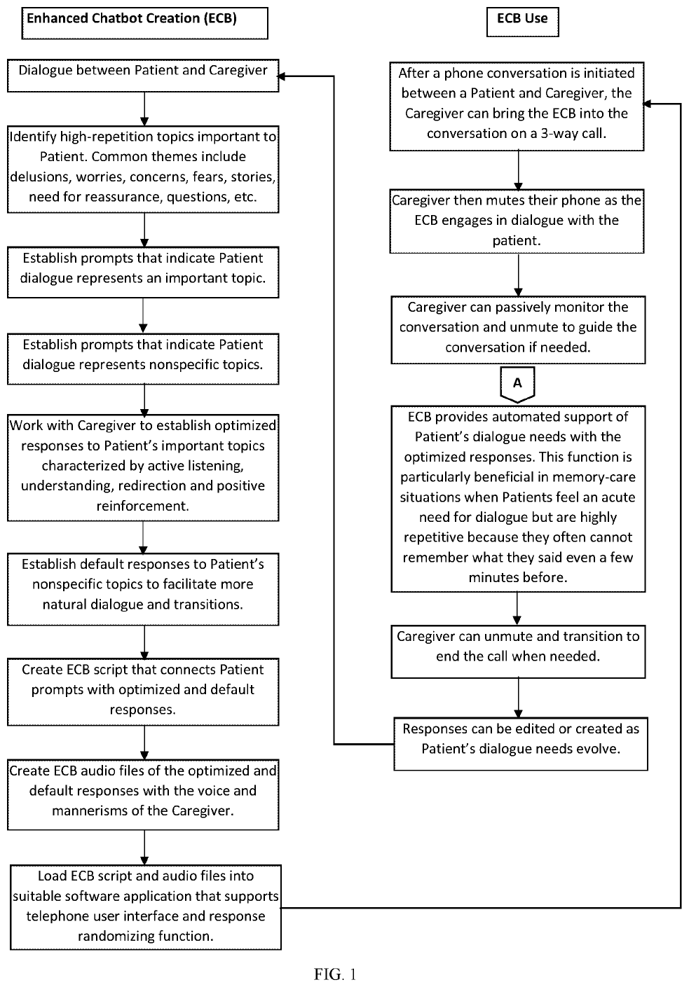

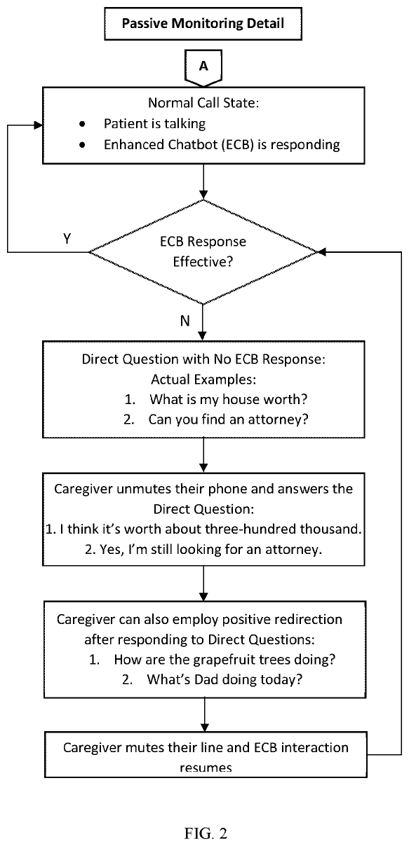

The exemplary and non-limiting example of a script below can form a part of systems according to certain embodiments of the subject invention. The script can also be used with methods according to certain embodiments of the subject invention, including but not limited to those exemplified in FIGS. 1-2.

TABLE 2
Functional Script

| | |
|---|---|
| Title | Are you there? |
| Prompts | Hear me? |
| | Are you still here? |
| | Are you still there? |
| | Are you there? |
| Responses | Yeah I'm here I'm just listening. |
| | Yeah I'm still here. |
| | Yeah I'm here, hey by the way, are you going to have grapefruit this year? |
| | Yep, still here, are they still working on the pipeline? |
| Audio | Imhere.wav |
| Database | Imhere2.wav |
| | Imheregrapefruit.wav |
| | Imherepipeline.wav |
| Title | Bothering you? |
| Prompts | Sorry I'm bothering you. |
| | Sorry I bothered you |
| Responses | It's never a bother to talk to you, mom. How's Casey doing? |
| | You're not bothering me mom, I love you! |
| Audio | nobothercasey.wav |
| Database | nobother.wav |
| Title | Can you call Ryan? |
| Prompts | Can you call Ryan? |
| Responses | Don't worry mom, I already told Ryan everything |
| Audio | toldryaneverything.wav |
| Database | |
| Title | Did I tell you this? |
| Prompts | Did I tell you this? |
| | Did I tell you this already? |
| Responses | Yes |
| | Uh, yeah |
| Audio | Yes 2.wav |
| Database | Uh yeah.wav |
| Title | Edmund? |
| Prompts | Edmund? |
| Responses | Yeah mom I'm listening |
| | I'm here, mom! |
| Audio | yeahmomimlisng.wav |
| Database | imhere.wav |
| Title | I don't know . . . |
| Prompts | I don't know what to do. |
| | I don't know who else to talk to. |
| | I don't know who to go to. |
| | I need someone to help me figure this out. |
| Responses | Me and Ryan will help you mom. |
| | You can always talk to me about it. |
| | Ryan knows and he's helping |
| | Don't worry mom, me and Ryan are looking for all those people who took the |
| | money. |
| Audio | meandryanwillhelp.wav |
| Database | talktomeaboutit.wav |
| | ryanknow.wav |
| | tookmoney.wav |
| Title | I need help |
| Prompts | I need help. |
| Responses | We're doing everything we can to help, but you know how hard it is to track down |
| | that much money. |
| | Don't worry mom, me and Ryan are trying to track down all those people who |
| | took your money. |
| Audio | trackdownmoney.wav |
| Database | trackdownhelp.wav |
| Title | Money stolen |
| Prompts | Are you having money stolen from your account? |
| | Are you having money stolen? |
| Responses | No, we're ok. |
| | No, we didn't have any money stolen from our accounts. |
| Audio | moneystolen-ok.wav |
| Database | nomoney.wav |
| Title | No one is going to help |
| Prompts | No one is going to help us. |
| | Nobody is going to help me. |
| Responses | Me and Ryan will help you, Mom. |
| | Hey mom, we're trying to help, we're looking for more information wherever we |
| | can. |
| | Don't worry mom, me and Ryan are tracking down those people you told us took |
| | the money! |
| Audio | meandryanwillhelp.wav |
| Database | tryingtohelp.wav |
| | trackdownmoney.wav |
| Title | Nobody believes me |
| Prompts | Nobody believes me. |
| Responses | We do believe you mom, and we're looking for the evidence. |
| | I love you, mom! |
| Audio | webelieveyou.wav |
| Database | loveumom.wav |
| Title | Ryan hold |
| Prompts | I can't get a hold of Ryan. |
| | Ryan never answers the phone. |
| | I can't reach Ryan. |
| Responses | Ryan knows and he's helping. |
| | I know he sometimes doesn't get the calls because he's out traveling a lot. |
| | I've talked with Ryan about everything you've told me, and he's helping also. |
| Audio | ryanknows.wav |
| Database | ryantravel.wav |
| | italkryan.wav |
| Title | Did you (affirmative)? |
| Prompts | Did you write this down? |
| Responses | Yes |
| | Uh, yeah |
| | Yes, I did just what you told me. |
| Audio | Yes 2.wav |
| Database | Uh yeah.wav |
| | Yestold.wav |
| Title | Can they (negative)? |
| Prompts | Can they bug a cell phone? |
| Responses | No, they can't do that. |
| Audio | Nocant.wav |
| Database | |
| Title | What year is it? |
| Prompts | What year is it? |
| Responses | It's two-thousand twenty-two. |
| Audio | Year2022.wav |
| Database | |
| Title | First time hearing? |
| Prompts | Is this the first time you're hearing this? |
| Responses | No, you've already told me. |
| Audio | Alreadytold.wav |
| Database | |
| Title | Have I told you? |
| Prompts | Have I told you this? |
| | Have I told you this already? |
| | Did you get that? |
| | Do you remember? |
| Responses | Yes |
| | Uh, yeah |
| | Yes, I always remember what you tell me. |
| Audio | Alwaysrem.wav |
| Database | |
| Title | Did I lose you? |
| Prompts | Did I lose you? |
| Responses | No, I'm still here! |
| Audio | Stillhere.wav |
| Database | |
| Title | Lose Dogs |
| Prompts | Lost my three dogs. |
| Responses | It's really good that you still have Casey! |
| | I'm glad you still have Casey, she's a really good dog! |
| | Well, you still have Casey, is she on the beanbag? |
| Audio | Goodcasey1.wav |
| Database | Goodcasey2.wav |
| | Goodcasey3.wav |
| Title | House worth |
| Prompts | What's the house worth? |
| | What is my house worth? |
| Responses | Five-hundred thousand. |
| | Half-a-million, isn't that great? |
| | Ryan looked it up for you and he says five hundred thousand. |
| Audio | 500k.wav |
| Database | halfMM.wav |
| | RyanhalfMM.wav |
| Title | Can you? |
| Prompts | Can you . . . |
| Responses | Yeah, I'll see what I can do. |
| | Sure, I can do that if you want me to. |
| Audio | Yesican.wav |
| Database | Sureican.wav |
| Title | I love you. |
| Prompts | I love you. |
| Responses | I love you too, mom! |
| | I love you too, mom, it's good talking with you. |
| | Awww, I love you too, mom! |
| Audio | Loveu1.wav |
| Database | Loveu2.wav |
| | Loveu3.wav |
| Title | Did you get the money? |
| Prompts | Did you get money in your account? |
| | Did you get the money? |
| | Did you get any money? |
| Responses | I looked, but didn't see any extra money in our account. |
| Audio | Noexmoney.wav |
| Database | |

### Functional Script with Generative AI in Unidirectional Mode

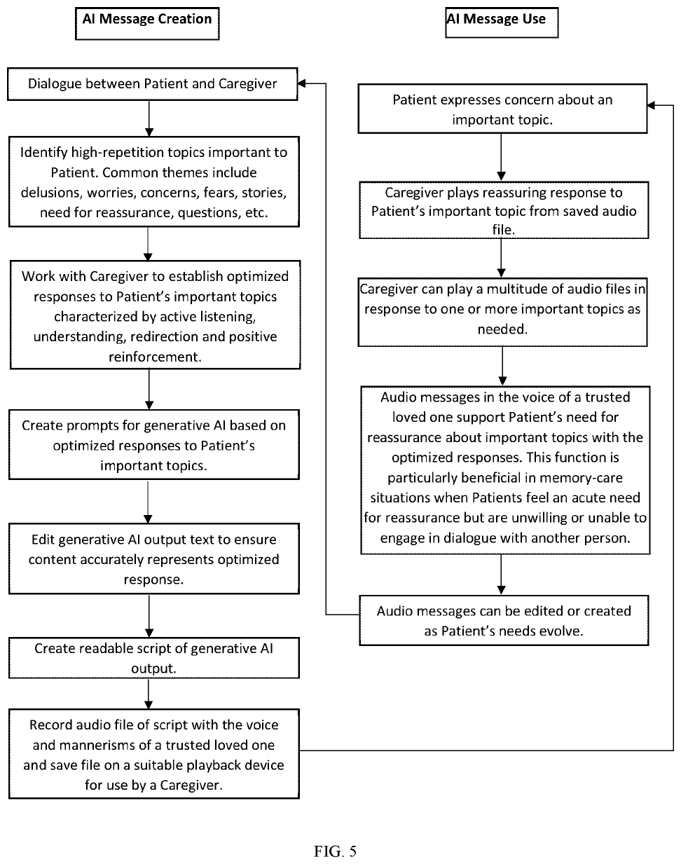

The exemplary and non-limiting example of a script below can form a part of systems according to certain embodiments of the subject invention. The script can also be used with methods according to certain embodiments of the subject invention, including but not limited to those exemplified in FIG. 5.

Embodiments employ Generative AI to create different types of content based on stylistic guidance given in the prompts, and this advantageously enables the efficient creation of abundant optimized responses for a given important topic. For example, a dementia patient expressing repetitive concerns about loss of their home and financial resources can be positively reassured by two different AI messages, one created in the manner of an “official report” and another created in the manner of a simple “voicemail”. The generative AI can create coherent responses such that the entire “official report” is written in an authoritative style, while the entire “voicemail” would sound more conversational. The positive message that the home and financial resources are fine would be the same in both examples, but the stylistic differences would provide the patient experience of having more sources of this positive news. Hearing the positive message from different perspectives can help the patient gain confidence that assuages their concerns.

Another example involves prompting the AI to create an “uplifting story” that involves a number of positive themes from the patient's life. Using just three input parameters requesting an uplifting story involving the patient, their devoted pet, and their farm, the provided AI is capable of creating a rich narrative (e.g., over 500 words in length), that can be converted to an audio file (e.g., of approximately 3 minutes.) This type of story (e.g., read in the voice of a trusted loved one) can substantially redirect the thoughts of a dementia patient from delusion-induced anxiety to positive consideration of an uplifting story where they are the main character. Generative AI, as applied by certain embodiments of the subject invention, can produce an abundance of varied content around simple, meaningful prompts derived from responses optimized for dementia patients, which provides a beneficial tool for assisting patients and caregivers.

### Example 2: AI-Generated Voicemail

Hey Mom it's Edmund. I just wanted to leave you a quick message to let you know that everything is alright and you don't need to worry. I know you've been concerned about your money and property and your house but I took the time to talk to all the necessary authorities to get some answers. I can promise that nobody is stealing your money, property or house. I double checked with the bank, the real estate agency, and even the property appraiser and they all confirm that everything is secure, your assets are safe, and there's no need to stress about it anymore. I also wanted to let you know that me, Ryan, Curtis and Brittany are doing just fine, we're all safe and healthy so please don't worry. You raised us well and we're making sure to stay safe. Please make sure to water your plants and take care of Casey, she's a good dog and you're really lucky to have her! Take care Mom, I love you!

### Example 3: AI-Generated Report

Hi Mom, I just wanted to share this report I got from Wells Fargo VIP account services that confirms everything is safe.

### Dear Ermida,

I hope this report finds you in good health and spirits I am writing to provide you with the latest updates on the matters you have expressed concern about. Your son Edmund has diligently undertaken the task of contacting the relevant authorities in conducting thorough investigations to ensure your Peace of Mind. I am delighted to inform you that after extensive communication with the authorities including law enforcement agencies and financial institutions it has been conclusively verified that no one is stealing your property, money, or house, your financial assets remain secure, and there is no indication of any unauthorized transactions or suspicious activities. Your property and home are also safe and there have been no reports of any attempted theft or trespassing. Furthermore, I can assure you that your other children Ryan, Brittany, and Curtis are safe and sound. Edmund has personally confirmed their well-being and reached out to them to ensure their safety they are all doing well and there are no immediate concerns regarding their health security or overall welfare. Please be assured that we remain vigilant in monitoring your financial matters and will continue to take all necessary precautions to protect your assets. We are here to support you in any way we can. In conclusion, all the necessary measures have been taken and I am pleased to confirm that your finances are secure and your children are safe. You can rest assured that everything is well taken care of. Warm Regards, Wells Fargo VIP Account Services

So this is really good news Mom! Everyone is working to take care of you and keep you safe. Just wanted to share this message with you. I'll come by to see you soon. Love you, bye!

### Example 4: AI-Generated Story

Hi Mom, I wrote a story based on all the times together around the farm and I wanted to share it with you, so I'm going to read it to you now, hope you like it!

Once Upon a time in a picturesque countryside there was a loving mother named Ermida who lived on a beautiful farm with her loyal and playful dog Casey. The farm was a haven of tranquility surrounded by lush woods, expansive pastures, a bubbling creek and fragrant grapefruit trees. Ermida was a woman full of joy and wisdom and her days were filled with a delightful tasks of tending to the land and nurturing the farms bountiful crops. The grapefruit trees in particular held a special place in her heart because they were grown from seeds that came from her mother's home in Miami Gardens. Each year when they bore their sweet and succulent fruits, she would share them with the neighbors and the local community. Her generosity and warmth made her beloved by everyone in the area. One Sunday morning as Ermida and Casey strolled through the woods they encountered a family of rabbits. The little rabbits were frightened and Ermida noticed that they seemed to be lost. With a compassionate heart she gently reassured them and offered them some of the carrots she had harvested from her vegetable garden. The rabbits, feeling safe in Ermida's presence, happily nibbled on the treats and Ermida smiled at the delightful sight. From that day on Ermida and Casey made it a daily routine to visit the rabbits leaving out small treats and ensuring they were safe and well fed. The rabbits soon became a part of their farm family, adding even more joy to their lives. But life on the farm wasn't always smooth sailing. One year an unexpected freeze struck threatening the grapefruit trees and all the other crops on the farm. Ermida worked tirelessly trying to save the trees and the livelihood they provided. With unwavering determination, she managed to keep the water warm and nursed the trees back to health. During this challenging time Ermida's church community came together to help her and each other. They shared resources, offered words of encouragement and it reminded them that tough times could be overcome through unity and compassion. As the years went by Ermida's farm flourished, becoming a symbol of hope, love, and resilience for the entire region. People from all around would visit to witness the enchanting grapefruit trees and experience the warmth of Ermida's hospitality. One day one of Ermida's students named Lily came to visit the farm. Lily had heard stories of Ermida's kind heart and wanted to meet her in person. Ermida welcomed Lily with open arms, and they spent the day exploring the woods and pastures and even enjoying some freshly squeezed grapefruit juice. Lily was inspired by Ermida's story and decided to plant her own fruit trees back in her town. She brought some saplings back with her and with the knowledge she had gained from Ermida she started her own little orchard. Soon more and more people in her town followed suit and a ripple effect of kindness and community support spread far and wide. As the years passed Ermida's farm became not just a place of beauty but a symbol of hope, resilience, and the power of compassion. People came from all over the world to learn from Ermida's wisdom and to experience the love she shared with everyone including her beloved dog, Casey. Ermida's legacy of nurturing the land, caring for others and fostering community connections lived on for generations, ensuring that her farm would forever be a place of inspiration and joy. And so, the tale of Ermida, the mother with a heart as big as her farm and her faithful companion Casey continues to be shared, inspiring people to be kind and resilient and to embrace the beauty of life that surrounds them.

So, I hope you like it Mom! It was a story that was based on you and your farm, and I look forward to seeing you soon. Love you Mom, bye!

1. A conversational management system configured and adapted to enhance a repetitive communication between a caregiver and a patient, the system comprising:
a multiplicity of topics extracted from a dialogue between the patient and the caregiver, the multiplicity of topics comprising:
a set of primary topics that are repetitive and important to the patient, and
a set of secondary topics that are repetitive and non-specific to the patient;
a multiplicity of prompts extracted from the multiplicity of topics, the multiplicity of prompts originating from the patient and comprising:
a set of primary prompts related to the set of primary topics, and
a set of secondary prompts related to the set of secondary topics;
a multiplicity of responses derived from the multiplicity of topics, the multiplicity of responses originating from an enhanced chatbot (ECB) and comprising:
a set of primary responses mapped to the set of primary prompts, and
a set of secondary responses mapped to the set of secondary prompts;
an ECB script that connects the set of primary prompts to the set of primary responses and connects the set of secondary prompts to the set of secondary responses;
an ECB audio database that contains the set of primary responses and the set of secondary responses in a voice recognizable to the patient as a trusted voice; and
a patient-optimized voice response software application configured and adapted to deliver selected content from the ECB audio database according to the ECB script, thus supporting a prompt-response dialogue with the patient to enhance the repetitive communication between the caregiver and the patient.

2. The system according to claim 1, the set of primary responses including two or more characteristics selected from the group consisting of active listening, understanding, redirection, and positive reinforcement.

3. The system according to claim 2, the set of secondary responses comprising clarification, repetition, and confirmation.

4. The system according to claim 1, the set of primary responses and the set of secondary responses each, respectively, comprising either interactive or automated input, or both, from the caregiver.

5. The system according to claim 4, the set of primary responses comprising interactive input from the caregiver.

6. The system according to claim 1, the set of primary responses consisting essentially of automated input from the caregiver, and the set of secondary responses consisting essentially of automated input from the caregiver.

7. The system according to claim 1, wherein the trusted voice comprises one or more mannerisms derived from the patient, a family member of the patient, or a friend of the patient; the one or more mannerisms comprising at least one of cadence, intensity, regional dialect, accent, and cultural reference.

8. The system according to claim 7, wherein the trusted voice is the voice of the caregiver.

9. The system according to claim 1, wherein the patient-optimized voice response software application comprises a telephone user interface and a response randomizing function.

10. The system according to claim 9, wherein the response randomizing function is configured and adapted to provide a different response selected from the set of primary responses to each of two successive prompts selected from the set of primary prompts.

11. A method for creating a conversational management system configured and adapted to support a prompt-response dialogue with a patient to enhance a repetitive telephonic communication between a caregiver and a patient, the method comprising:
identifying a multiplicity of topics during a dialogue between the patient and the caregiver;
identifying a set of primary topics that are repetitive and important to the patient;
identifying a set of secondary topics that are repetitive and non-specific to the patient;
extracting a set of primary prompts from the set of primary topics;
extracting a set of secondary prompts from the set of secondary topics;
establishing a set of primary responses mapped to the set of primary prompts;
establishing a set of secondary responses mapped to the set of secondary prompts;
creating an enhanced chatbot (ECB) script that connects the set of primary prompts to the set of primary responses and connects the set of secondary prompts to the set of secondary responses;
creating an ECB audio database that contains the set of primary responses and the set of secondary responses in a voice recognizable to the patient as a trusted voice; and
loading the ECB script and ECB audio database into a patient-optimized voice response software application, thereby providing the conversational management system configured and adapted to support the prompt-response dialogue with the patient to enhance the repetitive telephonic communication between the caregiver and the patient.

12. The method according to claim 11, the set of primary responses including two or more characteristics selected from the group consisting of active listening, understanding, redirection, and positive reinforcement.

13. The method according to claim 12, the set of secondary responses comprising clarification, repetition, and confirmation.

14. The method according to claim 11, the establishing of the set of primary responses and the establishing of the set of secondary responses each, respectively, comprising either interactive or automated input, or both, from the caregiver.

15. The method according to claim 14, the establishing of the set of primary responses comprising interactive input from the caregiver.

16. The method according to claim 11, the establishing of the set of primary responses consisting essentially of automated input from the caregiver, and the establishing of the set of secondary responses consisting essentially of automated input from the caregiver.

17. The method according to claim 11, wherein the trusted voice comprises one or more mannerisms derived from the patient, a family member of the patient, or a friend of the patient; the one or more mannerisms comprising at least one of cadence, intensity, regional dialect, accent, and cultural reference.

18. The method according to claim 17, wherein the trusted voice is the voice of the caregiver.

19. The method according to claim 11, wherein the patient-optimized voice response software application comprises a telephone user interface and a response randomizing function.

20. The method according to claim 19, wherein the response randomizing function is configured and adapted to provide a different response selected from the set of primary responses to each repetition of a prompt selected from the set of primary prompts.

21-30. (canceled)

31. A unidirectional communication management system configured and adapted to enhance a positive communication from a caregiver to a patient, the system comprising:
a multiplicity of topics extracted from a dialogue between the patient and the caregiver, the multiplicity of topics comprising:
a set of primary topics that are recurring and important to the patient, and
a set of secondary topics that are recurring and non-specific to the patient;
a multiplicity of responses derived from the multiplicity of topics, the multiplicity of responses originating from an enhanced chatbot (ECB) and comprising:
a set of primary responses mapped to the set of primary topics, and
a set of secondary responses mapped to the set of secondary topics;
an ECB script that connects the set of primary topics to the set of primary responses and connects the set of secondary topics to the set of secondary responses;
an ECB audio database that contains the set of primary responses and the set of secondary responses in a voice recognizable to the patient as a trusted voice; and
a patient-optimized voice response software application configured and adapted to deliver selected content from the ECB audio database according to the ECB script, thus delivering a unidirectional positive reinforcement communication to the patient.

32. The system according to claim 31, the set of primary responses including two or more characteristics selected from the group consisting of active listening, understanding, redirection, and positive reinforcement.

33. The system according to claim 32, the set of secondary responses comprising clarification, repetition, and confirmation.

34. The system according to claim 31, the set of primary responses and the set of secondary responses each, respectively, comprising either interactive or automated input, or both, from the caregiver.

35. The system according to claim 34, the set of primary responses comprising interactive input from the caregiver.

36. The system according to claim 31, the set of primary responses consisting essentially of automated input from the caregiver, and the set of secondary responses consisting essentially of automated input from the caregiver.

37. The system according to claim 31, wherein the trusted voice comprises one or more mannerisms derived from the patient, a family member of the patient, or a friend of the patient; the one or more mannerisms comprising at least one of cadence, intensity, regional dialect, accent, and cultural reference.

38. The system according to claim 37, wherein the trusted voice is the voice of the caregiver.

39. The system according to claim 31, wherein the patient-optimized voice response software application comprises a telephone user interface and a response randomizing function.

40. The system according to claim 39, wherein the response randomizing function is configured and adapted to provide a different response selected from the set of primary responses to each of two successive occurrences of a topic selected from the set of primary topics.

41. A method for creating a unidirectional communication management system configured and adapted to enhance a positive communication from a caregiver to a patient, the method comprising:
identifying a multiplicity of topics during a dialogue between the patient and the caregiver;
identifying a set of primary topics that are repetitive and important to the patient;
identifying a set of secondary topics that are repetitive and non-specific to the patient;
establishing a set of primary responses mapped to the set of primary topics;
establishing a set of secondary responses mapped to the set of secondary topics;
creating an enhanced chatbot (ECB) script that connects the set of primary topics to the set of primary responses and connects the set of secondary topics to the set of secondary responses;
creating an ECB audio database that contains the set of primary responses and the set of secondary responses in a voice recognizable to the patient as a trusted voice; and
loading the ECB script and ECB audio database into a patient-optimized voice response software application, thereby providing the conversational management system configured and adapted to deliver the unidirectional communication to the patient to enhance the positive communication from the caregiver to the patient.

42. The method according to claim 41, the set of primary responses including two or more characteristics selected from the group consisting of active listening, understanding, redirection, and positive reinforcement.

43. The method according to claim 42, the set of secondary responses comprising clarification, repetition, and confirmation.

44. The method according to claim 41, the establishing of the set of primary responses and the establishing of the set of secondary responses each, respectively, comprising either interactive or automated input, or both, from the caregiver.

45. The method according to claim 44, the establishing of the set of primary responses comprising interactive input from the caregiver.

46. The method according to claim 41, the establishing of the set of primary responses consisting essentially of automated input from the caregiver, and the establishing of the set of secondary responses consisting essentially of automated input from the caregiver.

47. The method according to claim 41, wherein the trusted voice comprises one or more mannerisms derived from the patient, a family member of the patient, or a friend of the patient; the one or more mannerisms comprising at least one of cadence, intensity, regional dialect, accent, and cultural reference.

48. The method according to claim 47, wherein the trusted voice is the voice of the caregiver.

49. The method according to claim 41, wherein the patient-optimized voice response software application comprises a telephone user interface and a response randomizing function.

50. The method according to claim 49, wherein the response randomizing function is configured and adapted to provide a different response selected from the set of primary responses to each repetition of a prompt selected from the set of primary prompts.

51-56. (canceled)

57. The system according to claim 31, wherein the patient-optimized voice response software application is based on a telephony system.

58. The method according to claim 41, wherein the conversational management system is based on a telephony system.

59-62. (canceled)

63. The system according to claim 1, wherein the patient-optimized voice response software application is based on a telephony system.
